(19) United States
(12) Patent Application Publication
(10) Pub. No.: US 2024/0400965 A1
(43) Pub. Date: Dec. 5, 2024

(54) PROCESS AND KIT TO INVESTIGATE MICROGRAVITY EFFECT ON ANIMAL/VEGETABLE CELLS UNDER EXTRATERRESTRIAL CULTIVATION CONDITIONS AND CULTIVATION PROCESS THEREOF TO SUSTAIN MANNED SPACE MISSIONS

(71) Applicants: UNIVERSITA' DEGLI STUDI DI CAGLIARI (20%), Cagliari (IT); UNIVERSITA' DEGLI STUDI DI SASSARI (20%), Sassasri (IT); TOLO GREEN S.R.L. (20%), Milano (IT); DISTRETTO AEROSPAZIALE SARDEGNA SOCIETA' CONSORTILE A R.L.(20%), Cagliari (IT); CENTRO DI RICERCA, SVILUPPO E STUDI SUPERIORI IN SARDEGNA - CRS4 SRL (20%), Pula (IT)

(72) Inventors: Giacomo Cao, Cagliari (IT); Antonella Pantaleo, Porto Torres (IT); Alessandro Concas, Cagliari (IT); Giacomo Fais, Carbonia (IT); Federico Micheli, Novi Ligure (IT); Alessia Manca, Porto Torres (IT); Gilberto Gabrielli, Milano (IT)

(73) Assignees: UNIVERSITA' DEGLI STUDI DI CAGLIARI (20%), Cagliari (IT); UNIVERSITA' DEGLI STUDI DI SASSARI (20%), Sassasri (IT); TOLO GREEN S.R.L. (20%), Milano (IT); DISTRETTO AEROSPAZIALE SARDEGNA SOCIETA' CONSORTILE A R.L. (20%), Cagliari (IT); CENTRO DI RICERCA, SVILUPPO E STUDI SUPERIORI IN SARDEGNA - CRS4 SRL (20%), Pula (IT)

(21) Appl. No.: 18/700,717

(22) PCT Filed: Oct. 13, 2021
(86) PCT No.: PCT/EP2021/078376

§ 371 Date: Apr. 12, 2024

(51) Int. Cl. C12M 1/42; B64G 7/00; C12M 1/00; C12M 1/34

(52) U.S. Cl. CPC C12M 35/04; B64G 7/00; C12M 21/02; C12M 41/34; C12M 41/40

(57) ABSTRACT

The present invention describes a technology to simulate extraterrestrial conditions for investigating the effect of microgravity and CO2 rich atmosphere on animal and vegetable cells cultivation and a process thereof exploiting extraterrestrial resources for producing edible biomass for the sustainment of manned space missions as well as the kit of materials and apparatus for implementing the same.

### FIELD OF THE INVENTION

The present invention refers to cultivation of vegetable cells under microgravity conditions and growth medium thereof to obtain protein-rich edible biomass for sustaining long-term manned space missions, in particular it further refers to apparatus for, or methods of, winning materials from extra-terrestrial sources and simulation thereof on earth. The present invention also refers to cultivation of animal cells under microgravity conditions with the said apparatus.

### STATE OF THE ART

The interest of several companies and institutions to undertake in the next 40 years manned missions on asteroids, Moon and Mars is well known. Specifically, within the framework of the current space exploration programs, the acronym ISRU (In Situ Resource Utilization) is typically accounted for. This acronym is related to the use of extra-terrestrial resources already available on Moon, Mars and/or asteroid which allows longer manned missions duration and cost reduction.

Most ISRU technologies consists of physico-chemical methods for oxygen and propellants production from Martian regolith and atmosphere, but cannot per se contribute to the production of the food necessary to feed crew members.

In such framework, novel technologies referred with the acronym ECLSS—Environmental Control and Life Support System—are being developed, for the production of food and water through recycling of liquid and solid wastes produced by astronauts involved in the research activities carried out on board the International Space Station (ISS).

Since 1988, with the aim of implementing ECLSS paradigms on the real scale, ESA (European Space Agency) is working at the project MELISSA (Micro Ecological Life Support System Alternative), which involves the use of microorganisms such as algae, bacteria and fungi, for the development of a closed loop process (i.e. producing all the materials needed by the crew only through recycling of waste and energy) to create, within the crew cabin, suitable conditions that allow its members to survive and work during long-term permanent missions on Moon and Mars.

Although the ultimate goal of the MELISSA project is to achieve a self-sustaining system, modeling simulations have shown that even the minimal goal of getting 20% of food required by crew members by means of waste recycling is not achievable through the current technology.

Similar results have been obtained through other ECLSS systems thus demonstrating that, at the state of the art, these systems are not completely self-sustaining and require the integration of external inputs of oxygen, food and water to meet the astronauts needs. In view of deep space manned mission such integrative resources cannot be continuously supplied from earth due to the related unaffordable mission cost and thus must be produced by exploiting in-situ available resources. However, production of food cannot avoid the exploitation of microorganisms and bioengineering techniques.

In this context, a recent field of research, known with the acronym bio-ISRU, is being developed to study the possibility of producing food in-situ by means of bioengineering techniques that involve the use of Martian regolith and atmosphere. Bio-ISRU technologies can be divided into two main categories, i.e. those ones relying on methylotrophic bacteria and using lithio-autotrophic microorganisms, respectively.

The first group of technologies is based on the use of methanol, which can be produced in-situ via physico-chemical ISRU, to obtain protein-rich edible biomass by means of microorganisms such as Pichia pastoris and Methylophilus methyloprophus or engineered Escherichia coli and Bacillus subtilis.

The second category of bio-ISRU processes is based on rock weathering microalgae or cyanobacteria which can photosynthetically convert N₂ and CO₂ available in the Martian atmosphere, along with S, P, Fe, Zn, Na and other micronutrients in the regolith, into suitable edible biomass by relying on the water and the light available in-situ. The use of cyanobacteria and microalgae leads to the further positive effect of producing photosynthetic oxygen which is crucial for the crew and can integrate the amounts produced via physico-chemical ISRU processes.

WO2013014606 describes a bio-ISRU process involving algae and cyanobacteria. The process comprises two main sections:
a “physico-chemical section” wherein CO₂, H₂O, N₂ and Ar are extracted, by means of WAVAR, TSA and MPO units, from the extra-terrestrial atmosphere and regolith and then used to produce O₂, H₂, CO, HNO₃, NH₃ and NH₄NO₃ via physico-chemical ISRU processes; and,
a “biological section” wherein edible microalgae and/or cyanobacteria from earth are cultivated in photobioreactor placed indoor of a dome, the dome heated at least at 10° C. and having an internal pressure of at least 0.8 bar of CO₂ produced in the physico-chemical section, the photobioreactor exposed to artificial or natural light and fed with a culture broth, HNO₃ and bubbling CO₂, the culture broth being the aqueous liquid phase of a slurry consisting of dehydrated extra-terrestrial regolith and H₂O acidified with HNO₃, wherein dehydrated extra-terrestrial regolith, H₂O, CO₂ and HNO₃ are those produced in the “physico-chemical section”.

One aim of the present invention is to provide a bio-SRU process improved and more efficient with respect to that described in WO2013014606.

Another aim of the present invention is providing a kit of material and method thereof to simulate on earth growth of cells under extra-terrestrial conditions in order to study the feasibility of a proposed bio-ISRU process during a long term manned space mission.

### DEFINITIONS AND ABBREVIATIONS

bio-ISRU: Bioengineering techniques for food production by In Situ Resource Utilization
ECLSS: Environmental Control and Life Support System
ESA: European Space Agency
ISS: International Space Station
ISRU: In Situ Resource Utilization
LSB: laboratory scale bioreactor
LSP: laboratory scale photobioreactor
MELISSA: Micro Ecological Life Support System Alternative
MPO: Microwave Pizza Oven
RPM: Random positioning machine
TSA: Temperature Swing Adsorber
WAVAR: WAter Vapor Adsorption Reactor

### SUMMARY OF THE INVENTION

Subject-matter of the present invention is an apparatus for simulating on earth the cellular growth under the extra-terrestrial conditions on a pre-determined extra-terrestrial location, said apparatus comprising:
an insulating jar mounted on a 3D clinostat or random positioning machine (RPM), said jar capable of containing at least one laboratory scale bioreactor (LSB), said jar provided with a manometer, a gas inlet and a gas outlet;
a cylinder for storing a gas simulating an extra-terrestrial atmosphere, the cylinder having an outlet fluidly connectable with the inlet of the jar.

Further subject matter of the present invention is a method of simulating on earth the cellular growth under extra-terrestrial conditions, said method comprising using the simulation apparatus as described above. In particular, the method of the invention for the cultivation of an edible microorganism comprises a step of:
preparing a culture medium by mixing a liquid regolith leachate obtained by leaching with acidified water an extra-terrestrial regolith simulant, with a diluted astronaut's urine simulant and micronutrients, wherein the micronutrients are those unavailable by ISRU on the extra-terrestrial location and known to be essential for the growth of the strain to be cultivated.

The simulation apparatus and method thereof of the invention were successfully used to simulate and investigate the cellular growth of a variety of vegetal and animal cell lines under simulated extra-terrestrial conditions.

Simulation experiments performed using the apparatus of the invention according to the method of the invention provided evidence that the biomass productivity is very much higher when using the operating conditions of the method of the present invention compared to the conventional operating conditions on Earth.

Further subject-matter of the present invention is a bio-ISRU process for producing photosynthetic edible biomass and oxygen for sustaining long-term manned extra-terrestrial missions; said process comprising:
preparing an extra-terrestrial growth medium by mixing a regolith leachate with diluted astronaut urines coming from a ECLSS and other micronutrients unavailable in situ brought from earth which are essential for the growth of the edible biomass;
loading a photobioreactor with the extra-terrestrial growth medium and with an inoculum of the edible biomass brought from earth.

Simulation experiments of Arthrospira platensis cultivation performed using the apparatus of the invention according to the method of the invention provided evidence that the biomass productivity is very much higher when using the operating conditions of the process of the present invention compared to the operating conditions described in WO2013014606. This evidence clearly demonstrates the relevant improvement provided by the present invention with respect to the state of the art. Simulation experiments provided evidence that the process of the invention is advantageously feasible allowing the production of food and oxygen on Mars.

Further subject-matter of the present invention is a food for astronauts comprising the edible biomass obtained by the process of the invention.

Further subject-matter of the present invention is a kit of material specifically adapted for implementing the process of the invention during a long-term manned space mission; said kit of material comprising:
a system for conveying diluted astronaut urines coming from a ECLSS to a container for preparing the extra-terrestrial growth medium;
the micronutrients which are essential for the growth of the edible biomass and are unavailable in the extra-terrestrial location.

### DETAILED DESCRIPTION OF THE INVENTION

According to the present invention the term extra-terrestrial conditions means conditions which can be found on Mars, Moon or an asteroid which imply microgravity and an atmosphere rich of CO₂.

According to the apparatus of the invention the jar is preferably a transparent jar and the at least one LSB is preferably a transparent Laboratory Scale Photobioreactor (LSP) and consists preferably of a flask made of transparent, non-cytotoxic, biologically inert, and nondegradable material capable to ensure the penetration of light needed by algae to perform photosynthesis. Preferably, virgin polystyrene should be used to this aim. The LSPs preferably should be provided with a vent cap made of high-density polyethylene. A 0.2 μm non-wettable membrane should be sealed to the cap, with the function of providing consistent, sterile gas exchange while minimizing the risk of contamination. These caps are thus useful to ensure the CO₂ needed by algae into the broth culture as well as the counter diffusion and the elimination of photosynthetic oxygen that, if accumulated in the liquid in too high concentration, could inhibit microalgae growth and promote photo-oxidation phenomena. The size of the LSP should be compatible with the size of the extra-terrestrial dome simulant and the sizes of the clinostat or RPM. The shape of this unit could be cylindrical or prismatic provided that they allow easier pouring and access to the flask for pipetting.

According to the apparatus of the invention the at least one clinostat or RPM must bestow the jar (and the sample within it) a motion characterized by a resulting acceleration vector, whose module has an average value over time that is close to zero. Clinostats and RPM are based on different principles. Clinostats rely on keeping the cells evenly suspended by continuously rotating the system to decrease sedimentation. This is obtained through rotating the sample on a plane along a circular trajectory. On the contrary, the Random Positioning Machine simulates microgravity by rotating the sample randomly around two axes of rotation. Accordingly, the sample is constantly reoriented, and the resulting time-averaged gravity acceleration is close to zero or it can be programmed to simulate as closely as possible the known gravity conditions of any extra-terrestrial location (e.g. it is known that gravity on Mars is approximately ⅓ of the gravity on Earth). Random positioning machine, rather than classic clinostats, should be preferred for algal cultures.

According to the apparatus of the invention the jar, functioning as extraterrestrial dome simulant, preferably consists of a cylindrical jar with sizes enough to contain at least one loaded LSP and such that it can be mounted on the clinostat or the RPM. It is preferably made of transparent material so to allow the transmission of light to the culture broth and permit photosynthesis to take place. The material preferably is also non-cytotoxic, biologically inert, and nondegradable. The jar should be provided with at least one inlet to be connected via suitable pipe to the cylinder containing the extra-terrestrial atmosphere simulant (preferably CO₂). The jar should be provided with an opening system which permit the easy insertion and discharge of the LSP at the end of the experiment. Moreover the dome is preferably provided with a manometer capable to measure the pressure in the jar.

The method of the invention, using the apparatus of the invention preferably comprises the following steps:
preparing a culture broth culture broth which is as close as possible to the optimal medium for the cell line to be grown, and when possible by simulating extra-terrestrial ISRU;
loading the LSP of the apparatus of the invention with the culture broth and successively with the inoculum of a microalgae or cyanobacteria strain to be cultivated;
arranging the LSP within the jar of the apparatus which simulates an extra-terrestrial dome;
mounting the jar on the clinostat or RPM of the apparatus of the invention;
connecting the outlet of the cylinder of the apparatus of the invention with the gas inlet of the jar;
insufflating the extra-terrestrial atmosphere simulant into the jar with the gas outlet of the jar open for a time sufficient to wash the jar inner atmosphere;
closing the jar outlet while keeping insufflating the extra-terrestrial atmosphere simulant until an internal pressure of at least 0.8 bar is achieved within the jar;
switching on the clinostat or RPM to simulate microgravity.

The method of the invention, using the apparatus of the invention for the cultivation of an edible biomass, preferably further comprises preparing a culture medium by mixing a liquid regolith leachate obtained by leaching with acidified water an extra-terrestrial regolith simulant, with a diluted astronaut's urine simulant and micronutrients unavailable by ISRU and essential for the growth of the strain to be cultivated.

The method of the invention, using the apparatus of the invention for the cultivation of an edible biomass, preferably comprises the following steps:
a) preparing an extraterrestrial regolith simulant;
b) contacting the extraterrestrial regolith simulant with a leaching solution to obtain a regolith slurry; the leaching solution being water acidified with HNO₃;
c) filtrating the regolith slurry to obtain a solid exhaust-regolith and a liquid regolith leachate.
d) preparing an astronaut's urine simulant;
e) dilute the urine simulant with water in order to simulate the dilution determined by flushing-water in most ECLSS. This way a ECLSS wastewater simulant is obtained.
e′) eventually further dilute the wastewater simulant if its salinity is too high to be compatible with microalgae growth.
f) preparing a culture medium with the optimal micronutrients for the growth of the strain to be cultivated;
g) mixing the ECLSS wastewater simulant with the regolith leachate and the culture medium to obtain a culture broth;
h) loading the LSP of the apparatus of the invention with the culture broth and successively with the inoculum of a microalgae or cyanobacteria strain to be cultivated;
i) arranging the LSP within the jar of the apparatus which simulates an extra-terrestrial dome;
j) mounting the jar on the clinostat or RPM of the apparatus of the invention;
k) connecting the outlet of the cylinder of the apparatus of the invention with the gas inlet of the jar;
l) insufflating the extra-terrestrial atmosphere simulant into the jar with the gas outlet of the jar open for a time sufficient to wash the jar inner atmosphere;
m) closing the jar outlet while keeping insufflating the extra-terrestrial atmosphere simulant until an internal pressure of at least 0.8 bar is achieved within the jar;
n) switching on the clinostat or RPM to simulate microgravity and simultaneously irradiating the dome simulant with natural or artificial light to promote photosynthesis.

Extraterrestrial regolith simulant can be prepared according to Planetary Simulant Database (https://simulantdb.com/) of the Colorado School of Mines. In case of Martian regolith simulant, it preferably should be the JSC Mars regolith or the Mojave Martian simulant whose chemical composition is provided by vendors or in the scientific literature (Peters, et al. Icarus. 197 (2008) 470-479. https://doi.org/10.1016/j.icarus.2008.05.004). Typically, the major elements in these simulants are silicon, aluminum, iron, magnesium, calcium etc., while the main crystalline phases can be attributed to magnetite, anorthite, hematite, forsterite, enstatite, wollastonite and others (Corrias, et al.; Acta Astronaut. (2012). https://doi.org/10.1016/j.actaastro.2011.07.022).

The leaching solution should be water acidified preferably by adding HNO₃ so to achieve a solution pH of at maximum 8.0. Then the regolith simulant and the resulting liquid should be inserted along with the leaching solution in a lab scale reactor provided with an agitation system. The solid-liquid weight ratio is preferably at least 1/10 g/g. The agitation levels should be preferably not lower than 100 rpm. Contact times of at least 24 hours should be ensured before proceeding to the next step. During this step (b) several micro-nutrients needed by algae to grow such as Fe, Zn, Ca, Na, Mg, are transferred from the solid to the liquid phase. The subsequent step c) consists of separating the liquid supernatant from the exhausted regolith simulant. It should be noted that the supernatant should be filtrated until a low turbidity level is achieved corresponding to a maximum optical density of about 0.05.

The step d) involves the preparation of a simulant of astronaut's urine according to the procedure by Sarigul et al. (Sci. Rep. 9 (2019) 1-11. https://doi.org/10.1038/s41598-019-56693-4). This step is crucial since urine produced by astronauts during manned missions on Mars or other extraterrestrial locations is one of the main source of nitrogen and phosphorous based macronutrients essential for the growth of microalgae. In fact, according to Sarigul et al. Sci. Rep. 9 (2019) 1-11. https://doi.org/10.1038/s41598-019-56693A) human urine is characterize by the composition shown the following parts of the document. Thus, it will be crucial to withdraw an amount of urine from ECLSS and using it to produce the “culture broth”. For the sake of hygiene, the urine is typically eliminated from the cabin crew along with suitable amount of flushing water. In this regard the step (e) consists of diluting the urine simulant with amount of water that simulate the flushing water that will be used in the ECLSS. Depending on the specific ECLSS being considered different volumes of flushing water can be used. In order to avoid osmotic shocks for microalgae, caused by the high salinity of urine, if the water dilution in the step e) is too low, a further dilution step is optionally foreseen in a step e′). The dilution ratio should be preferably equal to 1 part in volume of urine simulant per 10 parts in volume of water.

The culture medium of step (f) preferably is Zarrouk's medium in case the strain to be cultivated is Arthrospira platensis.

In step (g) ECLSS wastewater simulant, the regolith leachate and the culture medium are mixed preferably in proportion 1:1:1 v/v.

The inoculum preferably consists of a small amount of one of the following algal strains: Gloeocapsa strain OU 20, Leptolyngbya strain OU_13, Phormidium strain OU_10, Chroococcidiopsis 029; Arthrospira platensis; Synechococcus elongatus; Anabaena cilindrica; Chlorella vulgaris; Nannochloris Eucaryotum or genetically engineered strains thereof. However, due its higher nutritional properties for astronauts (Soni et al., (2021). Food Supplements Formulated with Spirulina. In Algae (pp. 201-226). Springer, Singapore, doi: 10.1007/978-981-15-7518-1_9) the inoculum should consist preferably of the strain Arthrospira platensis or Spirulina platensis.

When inoculated, the strain should be preferably in the exponential growth phase and the axenicity, i.e. the absence of bacteria, rotifers or different strain, should be guaranteed.

The step (h) involves the feeding of inoculum into the laboratory scale photobioreactor (LSP) where the “culture broth” produced as shown at point (g) have been previously fed. The amount of inoculum added to the unit volume of culture broth should be such that the optical density (at a wavelength of 650 nm) of the resulting solution is in the range 0.05-0.15.

The step (i) consists of arranging the LSP within a transparent insulated jar that simulates an extraterrestrial dome hosting a biological process wherein thermo-baric conditions close to the terrestrial ones are re-created. The jar must be transparent because the refracted light radiation should have an intensity of at least 30 μmol m⁻² s⁻¹, but preferably close to 100 μmol m⁻² s⁻¹, in order to promote an effective photosynthesis of microalgae. The latter one is the phenomenon underlying the growth and replication of algae that will be used as food as well as the production of the oxygen needed by crew members. However, algae necessitate also of a period of dark to perform the Calvin cycle reactions (the so-called dark reactions). For this reason, the light source should be switched off for at least 8 hours per day. Preferably the dome simulant is irradiated with a light bulb capable to provide a photosynthetic active radiation of intensity equal to at least 20 μE m⁻² s⁻¹, but preferably close to 100 μmol m⁻² s⁻¹, to the LSP. Preferably the light bulb is arranged close to the clinostat or the RPM. Preferably the light bulb is connected to a timer capable to switch on the light source for a photoperiod of 12 hours/day.

The step (j) consists of mounting the “extra-terrestrial dome simulant” containing the “loaded LSPs” on a clinostat or a random positioning machine (RPM)”. The latter ones, which will be better described in the kit section, are tools capable to recreate microgravity in the LSP through a 3-D motion characterized by acceleration whose temporal average is close to 0 (about 0.05 g) or as close as possible to the gravity of the extra-terrestrial location.

In the step (k) the extraterrestrial dome simulant is connected to a gas cylinder through a suitable port and a pipe. Preferably the gas-cylinder contains a gas simulating the extraterrestrial atmosphere, in case of simulating Mars atmosphere preferably the gas cylinder contains pure CO₂ The CO₂ is needed by microalgae to grow and represents a natural resource available on Mars or other extra-terrestrial locations. In fact, for example Martian atmosphere mainly consists (about 95% v/v) of CO₂ which can be separated from other gases and pressurized to be fed in the Martian Domes as reported in WO2013014606. Preferably a value of at least 0.8 bar is prescribed for the pressure of CO₂ within the dome. For this reason, the step (I) of the method of the invention preferably foresees the insufflation of CO₂ within the insulated jar until such pressure level is achieved. Recent papers (Verseux et al. 2021, Front. Microbiol. doi:10.3389/fmicb.2021.611798) have demonstrated that microalgae can thrive also under lower pressures of CO₂ therefore also lower pressures of CO₂ could be used. After the achievement of such pressure value the jar should be sealed tightly (step m).

The step (n) consists of the starting of the experiment by switching on the clinostat or the random positioning machine, switching on the lights and then waiting until at least 12 hours of light.

Preferably the Dome simulant should be emptied daily when the lights are switched off and kept empty for all the 12 hours of dark. Then when the lights are re-switched on, the Martian dome simulant should be re-loaded with CO₂ as described in the step (I) and sealed (step m). This sequence should be iterated for all the experiment duration.

This operation has to be done because CO₂ is not absorbed by algae when photosynthesis is not taking place (i.e. during dark periods) and thus too high concentration of CO₂ in the gas phase would lead to a unacceptably pronounced acidification of the culture broth which, in turn, would result in the algae growth inhibition. Then, after the 12 hours of dark period, the jar should be loaded with CO₂ as reported in the step (I) and sealed. Preferably, all the analysis needed to monitor the algae growth and other parameters should be performed during the dark phase when the light is off and the jar empty (without CO₂). The minimum set of parameters to be monitored involves, optical density, biomass concentration and pH concentration of the culture.

The apparatus and the method of the invention, similarly to what is described above about an edible microorganism cultivation, can be used to simulate the cellular growth under extra-terrestrial condition of any cell line. Cell lines whose growth can be simulated with the apparatus and method of the invention are all those from ECACC (https//www.phe-culturecollections.org.uk/products/celllines/generalcell/search.jsp?searchtext=hum an %20cell %20lines&dosearch=true) as well as those from ATCC (https://www.atcc.org/cell-products/animal-cells).

Further vegetable cell lines can be cultivated with the apparatus and method of the invention, preferably a cell line selected in the group consisting of Chlorella sorokiniana, Chlorella zofigensis, Coccomyxa sp., Synechococcus sp., Pseudochloris wilhelmii, Chlorella protothecoides, Euglena gracilis, Chlamydomonas reinhardtii, Isochrysis galbana, Neochloris oleoabundans Scenedesmus obliquus, Dunaliella salina, Nannochloropsis oculate, Chlorella pyrenoidosa, Botryococcus braunii, Phaeodactylum tricornutum, Tetraselmis sp., Thalassiosira pseudonana, Haematococcus pluvialis, Nannochloropsis oceanica, Spirulina maxima, Pavlova salina, Porphyridium marinum, Tetraselmis inconspicua, Cyanophora paradoxa, Thalassiosira rotula, Amphora sp., Odontella aurita, Attheya sp., Chromulina ochromonoides, Diacronema vlkianum, Chaetoceros sp., Navicula pelliculosa, Odontella mobiliensis, Porosira pseudodenticulata.

Further cell lines whose growth under extra-terrestrial condition can be simulated using the apparatus and the method of the invention are human and animal cell lines selected in the group consisting of:

H1, H9, Embryonic stem cells, Human; HEK-293, Embryonic kidney transformed with adenovirus, Human; HeLa, Epithelial cell, Human; HL 60, Human, promyelocytic leukemia cells, Human; MCF-7 Breast cancer, Human; A549, Lung cancer, Human; A1 to A5-E, Amnion, Human; ND-E, Esophagus, Human; CHO, Ovary, Chinese hamster; 3T3, Fibroblast, Mouse; BHK21, Fibroblast, Syrian hamster; MDCK, Epithelial cell, Dog; E14.1, Embryonic stem cells (mouse), Mouse; COS, Kidney, Monkey; DT40, Lymphoma cell, Chick; S2, Macrophage-like cells, Drosophila; GH3, Pituitary tumor, Rat; L6, Myoblast, Rat; Sf9 and Sf21, Ovaries, Fall Army worm; (Spodoptera frugiperda) ZF4 and AB9 cells, Embryonic fibroblast cells, Zebrafish; 1184, skin fibroblast, Human; E6.1 clones, Jurkat cell, Human; THP1 cells, Human; SH-SY5Y, Neuroblastoma cells, Human; iPSCs, stem cells, Human; BRISTOL 8, B lymphocyte, Human.

According to the apparatus and method of the invention most preferred cell line to be cultivated is selected in the group consisting of Erythrocyte cell culture, Human; C20A4, Chondrocyte cell, Human; 1301, T-cell leukemia, Human; 1306,161 BR, skin fibroblast, Human; F-36P myelodysplastic syndrome, leukemia, Human; H9, T-cell, Human; HeLa, Epithelial cell, Human; E6.1 clones, Jurkat cell, Human; SH-SY5Y, Neuroblastoma cells, Human; iPSCs, stem cells, Human; 1184, skin fibroblast, Human; hMSCs, mesenchymal stem cells, Human; mBMSC, Bone Marrow derived mesenchymal stem cell, Rat; ADSCs, Adipose-derived stem cell, Human; mESCs, Embryonic stem cells, Mouse; MG-63, Osteosarcoma cell lines, Human; HUVEC, Human umbilical vein endothelial cells, Human.

According to the method of the invention each cell line is cultivated in a culture medium which is as close as possible to its optimal medium, and when possible by simulating extra-terrestrial ISRU.

For simulating the growth of human and animal cell under extra-terrestrial conditions it is preferably performed the method as above described and steps a-g can be replaced by a step of preparing a culture broth which is as close as possible to the optimal medium for the cell line to be grown, and when possible by simulating extra-terrestrial ISRU; and irradiation in step n) can be omitted.

The process and the kit related to the present invention operates in connection with ECLSS sections necessarily present on the extra-terrestrial location during a long-term manned space mission, thus representing its ideal completion to the aim of achieving a self-sustaining integrated system. Therefore, the process is based upon the exploitation of extra-terrestrial resources such as the atmosphere, the soil and solar radiation, whose main features are reported in the literature, e.g. Nanagle [Nat. Biotechnol. (2020). https://doi.org/10.1038/s41587-020-0485-4] and Rapp [https://doi.org/10.1007/978-3-319-72694-6]. In particular, relatively high amounts (about 9% wt/wt) of hydration water has been detected in the Martian soil (Rieder, R., et al. Science 306, 1746-1749 (2004)).

The process of the invention preferably comprises the following steps:
a′. assembling on the extra-terrestrial soil at least one geodesic dome and place at least one photobioreactor within the dome;
b′. assembling a physico-chemical section comprising photovoltaic panels, at least a WAVAR unit, at least a TSA unit and at least a MPO unit for extracting from extra-terrestrial soil and atmosphere water, dehydrated and pressurized CO₂, N₂ and Ar and producing by NH₃, O₂, H₂, HNO₃, NH₄NO₃;
c′. blowing heated, pressurized and dehydrated CO₂ produced in the step (b′) within said dome until a pressure of at least 0.8 bar and temperature of at least 10° C., preferably between 1° and 15° C., is reached within the dome;
d′. preparing a leaching solution by mixing water and HNO₃ produced in the physico-chemical section;
e′. leaching the dehydrated regolith from the physico-chemical section with the leaching solution, preferably with a solid/liquid weight ratio of 1:5 for at least one Martian day (sol);
f′. filtering the regolith slurry to obtain a regolith leachate and leached regolith;
g′. preparing an extra-terrestrial growth medium by mixing a regolith leachate with diluted astronaut urines coming from a at least one ECLSS section, HNO₃ produced in the physico-chemical section and other micronutrients unavailable in situ brought from Earth which are essential for the growth of the edible biomass;
h′. preparing an inoculum of the edible microalgae or cyanobacteria brought from Earth;
i′. feeding the photobioreactor with the extra-terrestrial growth medium and successively with the inoculum to obtain a biological slurry;
j′. exposing the biological slurry to the CO₂ within the dome and to a light source which is capable of promoting photosynthesis, thus leading to the formation of new biomass algal and photosynthetic oxygen;
k′. separating the algal biomass from the spent “culture broth” by centrifugation and extracting photosynthetic oxygen by degassing;
l′. sending the oxygen to the ECLSS section and further dehydrating the algal biomass in order to use it as food or dietary supplement along with the food produced in the ECLSS section;
m′. splitting the spent “culture broth” into two streams named α₁ and α₂.
n′. recirculating the stream of the spent culture broth α₁ into the at least one photobioreactor.
o′. optionally conveying the stream α₂, together with the ammonium nitrate (NH₄NO₃) produced in the physico-chemical section, together with fresh regolith, together with suitable amounts of humic and fulvic acids brought from Earth, together with human metabolic wastes from ECLSS, into the domes where vegetables are grown.

The extraction processes with the physico-chemical section preferably involve the following steps:
b′-i. assembling outdoor photovoltaic panels which produce the energy required to heat the inner of the dome as well as to power said plant units;
b′-ii. assembling outdoor a Temperature Swing Adsorber unit (TSA);
b′-iii. assembling outdoor a the WAVAR unit for Mars atmosphere dehydration;
b′-iv. assembling outdoor the at least one Microwave Pizza Oven;
b′-v. blowing Mars atmosphere into the WAVAR unit, which operates outdoor to extract water from the atmosphere;
b′-vi. conveying Martian atmosphere to the TSA units where separation and pressurization of CO₂ is carried out through cycles of adsorption-desorption onto zeolitic materials at variable temperatures. In TSA units a secondary gas stream, consisting mainly of N₂ and Ar, is also produced;
b′-vii. storing said secondary gaseous stream of N₂ and Ar, produced as shown in step (g′), into suitable tanks from which it can be withdrawn to be exploited as buffer gas for the analytical equipment which will be employed during the sampling campaigns that will be performed for scientific purposes, during the mission;
b′-viii. heating the CO₂ blown into the at least one dome, through heating systems powered by said photovoltaic panels, until a temperature not lower than 10° C. is achieved within the dome;
b′-ix. excavating Martian regolith and conveying it to the MPO system which operates indoor and extracts adsorbed and hydration water from the regolith, by means of microwaves.

In particular, the WAVAR and TSA units will operate outdoor. These units, while operating under extra-terrestrial thermo-baric conditions, will be preferably mechanically protected by suitable structures from potential damage provoked by hits of meteorites and/or solids transported during the usual dust storms that characterize the extra-terrestrial environment. Such structures may be obtained in situ through specific technologies such as, for example, the technology described in WO 2012/014174 A2.

The MPO unit operates indoor.

The process of the present invention preferably involves a first step (a′) where, the domes within which the plant units operating indoor that are needed for implementing the process are installed and assembled. Inside the domes, by means of techniques better specified in what follows, thermo-baric conditions (temperature and pressure) are set at which the state of aggregation of reactants and products is completely analogous to the one observed on earth for the same compounds.

Step (e′) preferably involves the feeding of a regolith stream flow together with a stream of nitric acid, together with a water stream into a reactor where liquid and solid are contacted in order to form a slurry which is continuously stirred thus allowing an effective contact between liquid and solid phase. The goal of such step is to transfer all macronutrients (P, S, C) and micronutrients (Fe, Mg, Si, etc.) contained by the regolith, to the liquid phase. This way, a “regolith leachate” is produced which, once integrated with other nutrients, will be able to sustain autotrophic algal growth phenomena. Preferably the contact time to ensure an effective mass transfer of nutrients into the liquid phase is about 24 hours.

Step (f′) of the process involves the solid/liquid separation which can be carried out by means of suitable filtrating systems (i.e. filter plates or filter bags).

Therefore, the operating step (f′) generates two separated streams, the first one is the leached regolith while the second one is a liquid called “regolith leachate”. The latter one is mixed with astronaut's urine, diluted with flushing water, coming from ECLSS to obtain a solution containing suitable nutrients for the biomass. In fact, human urine typically contains crucial macronutrients such as ammonium, nitrates, phosphates, and orthophosphates which are in general the limiting factors for biomass growth. So, the use of this metabolic waste can greatly improve the capability of the resulting medium to sustain microalgae growth. Minimal amounts of other nutrients unavailable in situ, needed to obtain a balanced growth medium could be brought from Earth.

Preferably the inoculus consists of the following algal strains: Gloeocapsa strain OU_20, Leptolyngbya strain OU_13, Phormidium strain OU_10, Chroococcidiopsis 029; Arthrospira platensis; Synechococcus elongatus; Anabaena cilindrica; Chlorella vulgaris; Nannochloris Eucaryotum or genetically engineered strains thereof. However, due to its higher nutritional properties, the strain Arthrospira platensis should be preferred.

The step (i′) involves the feeding of the inoculum in a photobioreactor where the “Martian growth medium” is simultaneously fed. The resulting mixture within the photobioreactor will be hereafter named “biological slurry”. According to step (j′) the CO₂ needed by the biomass to perform photosynthesis is taken from the atmosphere consisting of pure CO₂ within the dome through suitable openings in the photobioreactor preferably covered by a semi-permeable membrane that allows the diffusion of CO₂ towards the biological slurry and the counter diffusion of oxygen produced by photosynthesis. The latter one is carried out by algae thank to the light flux provided by the light source according to step (j′) of the process. The light flux can be supplied by directly exposing the culture to the solar radiation incident on Martian surface or, preferably, by means of suitable systems such as light concentrators and optical fibers. Therefore, the photosynthetic process leads to the production of new microalgae which results in the increase of algal biomass concentration in the culture.

According to a preferred embodiment, photobioreactors operate in fed-batch mode. Therefore, biomass cultivation is carried out in the photobioreactor until biomass concentration reaches suitable values which correspond to the stationary phase of the growth kinetics of the biomass. Once the stationary phase is reached, a suitable amount of “biological slurry” is withdrawn and undergone to dehydration processes for separating the biomass from the spent “culture broth”. The amount of biological slurry that was withdrawn from the photobioreactor is then replaced by an equal amount of fresh “culture broth” which re-supplies the nutrients consumed during biomass growth. An aliquot of medium needed to replace the withdrawn amount of biological slurry can be also obtained by recirculating the solution according to step (n′). Once the operating steps of withdrawal and reintegration of fresh “culture broth” are performed, micro-algae growth is restarted again in batch mode. The operations of removing and re-integrating should be repeated periodically, preferably once for day, at the same hour of the day, in order to ensure for example at least 25 hours (duration of the Martian day) of growth in batch mode.

The step (k′) of the process involves the transfer of the “biological slurry” extracted daily, to a step of solid-liquid separation that is carried out by means of suitable centrifugation systems. The solid-liquid separation performed in this step allows separating the algal biomass from the spent “culture broth”.

The latter one can be recirculated to the head of the photobioreactor in order to reduce the inlet amounts of water needed. Another aliquot of spent culture broth, which contains residual contents of relevant nutrients, according to a preferred embodiment, can be used for irrigation purposes in the greenhouses of the process described in WO2013014606.

The solid algal biomass separated by centrifuges, can be further dehydrated by means of microwave ovens and then used as food by astronauts.

In step (l′), oxygen produced by the biomass by photosynthesis is separated from biological slurry through suitable degassing systems and then transferred to the ECLSS units where it can be used for the crew cabin air revitalization.

Simultaneously, microalgal biomass can be further dehydrated and then sent to the ECLSS to be used as food for astronauts. It is therefore a further subject-matter of the present invention a food for astronauts comprising microalgal biomass obtained by the process of the invention.

The step (o′) involves the transfer of the different products of the processes so far described within a dome which operate as greenhouse where plants and vegetables can be cultivated.

The kit of material of the invention preferably comprises:
at least one geodesic dome for housing the different plant units used in the physico-chemical group of the procedure above;
at least one photovoltaic system for producing the energy needed for heating the inner atmosphere of the at least one dome, as well as the energy needed for powering plant units operation;
at least one WAVAR unit based on the use of zeolites through which adsorption processes, followed by desorption through microwaves heating, are performed for the extraction of water from Martian atmosphere;
at least one TSA unit consisting of at least one adsorbent bed of zeolite and at least one radiator that ensure the heat exchange with Martian environment and the implementation of adsorption-desorption cycles at variable temperature that, in turn, allow the separation of CO₂ from other gases constituting the Martian atmosphere (mainly N₂ and Ar) as well as its pressurization. Pressurized pure CO₂ produced by TSA units can be the blown within the at least one dome until a suitable pressure is achieved in the inner of the dome;
at least one excavator and at least one conveyor belt for excavating and conveying the Martian regolith to the following treatment units;
at least one MPO unit, including at least one magnetron, for the extraction of adsorbed and hydration water from the Martian regolith by microwave heating;
at least one unit for mixing the water extracted from the regolith with suitable amounts of nitric acid produced in the physico-chemical section;
at least one leaching reactor operating in continuous mode, for leaching regolith through a mixture of water and nitric acid;
at least one unit consisting of a “filter plates” for the solid/liquid separation of the slurry stream outgoing from the leaching reactor. Said unit produces a liquid stream named “regolith leachate” and a solid stream of “leached regolith”;
at least one unit for mixing the “regolith leachate” with the urine diluted with flushing water produced by astronauts in the ECLSS to obtain the so called “culture broth”;
at least a tank for storing the gas, consisting mainly of N₂ e Ar, which has been produced in the TSA unit as a result of CO₂ separation;
at least one among the following algal strains: Gloeocapsa strain OU_20, Leptolyngbya strain OU_13, Phormidium strain OU_10, Chroococcidiopsis 029; Arthrospira platensis; Synechococcus elongatus; Anabaena cilindrica; Chlorella vulgaris; Nannochloris Eucaryotum or genetically engineered strains thereof.
at least one unit for preparing the inoculus of algal strains;
at least a photobioreactor for producing algal biomass;
nutrients from Earth, said nutrients those unavailable by ISRU but essential for the growth of the at least one algal strain and;
at least one unit for the separation of algal biomass and of oxygen produced in the photobioreactor from the spent “culture broth”;
at least one unit for dehydrating algal biomass; and
(optional) at least one geodesic dome to be used as a greenhouse for growing edible plants.

Preferably the domes are made by a framework of aluminum beams with a circular section. Preferably, the coverage of the geodesic dome is made by sheets of ETFE (Ethylene Tetrafluoroethylene) having a surface density of 0.2 kg/m² as well as a high mechanical and thermal resistance.

Preferably, the at least one photovoltaic system produces the energy that is needed to power all the operating steps of the invented process, including the step of heating the inner atmosphere of the domes. Under the electrical point of view, said photovoltaic system is preferably divided into separate sections (arrays), each of them having a surface of about 40 m², as well as a yield of converting solar radiation into electricity that is about 11%.

The use of TSA, which exploits adsorption/desorption cycle at variable temperature on zeolites, is proposed for separation, blowing and compression of CO₂ within the domes according to the principles previously described for TSA units. A suitable unit for the extraction of water from Mars atmosphere could be the one described by Williams, J. D., et al. (Journal of British Interplanetary Society, 1995, 48, 347-354).

The at least one excavator and at least one belt conveyor must excavate and transport the extraterrestrial regolith to the MPO units. The excavator consists of a vehicle powered by photovoltaically rechargeable batteries or independently by means of small photovoltaic systems housed on the same vehicle.

The at least one slurry reactor should be agitated and coated with anti-acid paint. Preferably the reactor size must be such to provide a residence time of at least 24 hours. The slurry outgoing from the reactor is transferred to the step where the solid/liquid separation takes place. To this aim the process of the present invention involves the use of at least one filter for separating the solid phase of the slurry from the liquid one.

Different typologies of photobioreactors may be employed, while tubular ones should be preferred. Tubes should be made of PET (polyethylene terephthalate) since they must be transparent with respect to the photosynthetically active radiation. Preferably tubes must be less than 0.2 m in diameter. Photobioreactor should be mainly operated in fed-batch mode. Light flux, necessary to promote photosynthesis, can be supplied through direct exposition the photobioreactor to the solar radiation incident on the extra-terrestrial surface or, preferably, by means of suitable light collecting systems such as light concentrators and optical fibers which transfer the light to the domes where photobioreactors are housed.

The nutrients from Earth, in case the kit include Arthrospira platensis algal strain preferably are boric acid and a complexing agent; more preferably H₃BO₃ and EDTA.

The amount of “biological slurry” that is withdrawn periodically from the photobioreactors should then undergone to a solid/liquid separation. Preferably the units for separating algal biomass from spent “culture broth” is carried out by means at least one centrifuge.

### BRIEF DESCRIPTION OF THE FIGURES

FIG. 1—scheme of the apparatus of the invention

FIG. 2—a graph of the gravitational acceleration achieved through the clinostat

FIG. 3—shows the time evolution algal biomass concentration during specific experimental trials aimed to isolate the effect of each operating conditions of the process with respect to the base case experiment Zm_Air_1 g: (A) effect of the use of Martian Medium; (B) effect of using simulated Martian atmosphere; (C) effect of microgravity. In figure (D) the effects of the synergic combination of two out of three of operating conditions of the invented process with respect to the base case conditions are shown.

FIG. 4—shows the effects of simulating the operating conditions to implement on Mars, according to the invented process (Mm40_CO₂_μg), in terms of (A) biomass concentration evolution in time and (B) final biomass productivity after 22 days of cultivation.

FIG. 5—shows the comparison between the results obtained under the operating conditions simulating the invented process (Mm40_CO₂_μg) and the ones simulating the process WO2013014606 of existing state of the art (RL_CO₂_μg), in terms of (A) biomass concentration evolution in time and (B) final biomass productivity after 22 days of cultivation.

FIG. 6—shows a flow-sheet of the process according to the Example of the present invention

### EXPERIMENTAL SECTION

### 1. Materials and Methods

### 1.1. Microorganism Maintenance Conditions

Unialgal culture of cyanobacterium Spirulina was obtained from algal cultivation TOLO green farm of Arborea, Sardinia, Italia. The strain was maintained at the laboratory of the Interdepartmental Center of Environmental Sciences and Engineering (CINSA), University of Cagliari, Sardinia, Italy, under axenic conditions. The cultures were kept in 250 mL Erlenmeyer flask, containing 150 ml Zarrouk's medium (Table 1) sealed with an aluminium foil that covered cotton wool.

TABLE 1
Composition of the Zarrouk' medium

| Components | (g/L) |
|---|---|
| NaCl | 1.00 |
| EDTA | 0.08 |
| FeSO₄ × 7H₂O | 0.01 |
| MgSO₄ × 7 H₂O | 0.20 |
| NaHCO₃ | 16.80 |
| CaCl₂ × 2H₂O | 0.04 |
| K₂HPO₄ | 0.50 |
| NaNO₃ | 2.50 |
| K₂SO₄ | 1.00 |

| Micronutrients | (mg/L) |
|---|---|
| H₃BO₃ | 2.86 |
| Na₂Mo₇O₄ | 0.0177 |
| CuSO₄ × 5H₂O | 0.079 |
| ZnSO₄ × 4H₂O | 0.222 |
| MnCl₂ × 4H₂O | 1.81 |

Flasks containing the culture media were autoclaved at 121° C. for 15 min prior to inoculation. The cultures were kept under photoautotrophic conditions and incubated in a thermostatically controlled chamber at 20±1° C. The photoperiod was fixed at 12:12 hours light and dark periods with white light illumination of 25 μmol/m²/s (Lightmeter Delta OHM HD 2302.0). Stirring was set at 100 rpm.

### 1.2. Preparation and Composition of the Martian Medium (MM)

MM was prepared by mixing a leachate of Martian regolith simulant (JSC MARS-1) and synthetic human urine (MP-AU) according to the literature (Sarigul, et al. Sci. Rep. (2019) https://doi.org/10.1038/s41598-019-56693-4). Main constituents of JSC MARS-1 used in the present study are reported in Table 2 in terms of oxides wt %. The mineral phases of JSC MARS-1 identified by XRD analysis consist primarily of tectosilicate plagioclase feldspar, pyroxene, iron oxides (magnetite and hematite), ilmenite and olivine (Peters, et al. Icarus. 197 (2008) 470-479. https://doi.org/10.1016/j.icarus.2008.05.004).

TABLE 2
Composition of Martian Regolith simulant
JSC Mars-1A in terms of oxides.

| Major element composition^{a} | wt % |
|---|---|
| Silicon dioxide (SiO₂) | 34.5-44 |
| Titanium dioxide (TiO₂) | 3-4 |
| Aluminum oxide (Al₂O₃) | 18.5-23.5 |
| Ferric oxide (Fe₂O₃) | 9-12 |
| Iron oxide (FeO) | 2.5-3.5 |
| Magnesium oxide (MgO) | 2.5-3.5 |
| Calcium oxide (CaO) | 5-6 |
| Sodium oxide (Na₂O) | 2-2.5 |
| Potassium oxide (K₂O) | 0.5-0.6 |
| Manganese oxide (MnO) | 0.2-0.3 |
| Diphosphorus pentoxide (P₂O₅) | 0.7-0.9 |

^{a}The normal convention for data presentation uses oxide formulae from an assumed oxidation state for each element (with the exception of Fe) and oxygen is calculated by stoichiometry.

The leachate was prepared by contacting 15 g of regolith simulant (<1 mm diameter size) with 150 ml of ultrapure water having a pH 6.80 within a 250 ml Erlenmeyer flask with cap. The solid liquid mixture was stirred at 200 rpm through an orbital shaker (Stuart SSM1, Bio sigma) for 24 hours at 25° C. The resulting solution was filtered by gravity by means of bibulous paper. Analysis of the supernatant was carried out using an inductively coupled plasma optical emission spectrometry (Varian 710-ES ICP OES) for the determination of Al, Ca, Fe, K, Mg, Mn, Na, P, Si, and Ti. The obtained results are shown in Table 3.

TABLE 3
Heavy Metals and micronutrients in
the JSC Mars-1A simulant leachate

| Metal | (mg/L) |
|---|---|
| Al | 4.80 ± 0.80 |
| Ca | 8.12 ± 0.52 |
| Fe | 6.41 ± 1.10 |
| K | 8.32 ± 0.40 |
| Mg | 1.48 ± 0.17 |
| Mn | 0.19 ± 0.03 |
| Na | 4.66 ± 0.32 |
| P | 0.25 ± 0.02 |
| Si | 10.28 ± 1.41 |
| Ti | 1.27 ± 0.23 |

Operating conditions were radiofrequency generator power 1.2 kW, frequency 40 MHz; Ar (99.996% purity) was used both for plasma (15 L/min), nebulizer (200 Kpa), and optic supply (1.5 L/min). The spray chamber was a double-pass, glass cyclonic. The power and pressure applied were 600 W and 100 PSI for 13 min. Calibration curves were calculated on five points and were considered acceptable for R²≥0.999. Synthetic human urine (MP-AU) was produced according to the literature Sarigul, et al. Sci. Rep. (2019) https://doi.org/10.1038/s41598-56693-4) and then diluted with ultrapure water at a ratio of 1:10 in order to meet the nitrogen requirement of microalgae. The chemical composition of diluted human urine simulant is shown in Table 4.

Finally, one-part of the leachate of Martian regolith and one-part of diluted urine were mixed (1:1 v:v) to produce the so-called Martian Medium (MM). Conductivity was 850 μS/cm and pH 7.4 at 25° C. Dilutions of MM (20, 40, 60 and 80%) were made whit Zarrouk's medium which was also the experimental control medium. MM and its dilutions have been sterilized at 121° C. for 15 min prior to use. Table 5 shows the composition of the resulting Martian Medium in terms of macronutrients while Table 6 shows its composition in terms of metals. Some of the metals, such as Zn, Fe, Mg, Si, Mn and K, can serve as micronutrients for algae.

TABLE 4
The composition of synthetic urine (MP-AU)*

| Components | (g/L) |
|---|---|
| Na₂SO₄ | 1.700 |
| C₅H₄N₄O₃ | 2.500 |
| Na₃C₆H₅O₇ × 2H₂O | 0.720 |
| C₄H₇N₃O | 0.881 |
| CH₄N₂O | 15.000 |
| KCl | 2.308 |
| NaCl | 1.756 |
| CaCl₂ | 0.185 |
| NH₄Cl | 1.266 |
| K₂C₂O₄ × H₂O | 0.035 |
| MgSO₄ × 7H₂O | 1.082 |
| NaH₂PO₄ × 2H₂O | 2.912 |
| Na₂HPO₄ × 2H₂O | 0.831 |

*C₅H₄N₄O₃ = huric acid; Na₃C₆H₅O₇ × 2H₂O = Sodium citrate dihydrate; C₄H₇N₃O = Creatinine; CH₄N₂O = Urea.

TABLE 5
Resulting composition of the Martian
Medium in terms of macro-nutrients

| Components | (g/L) |
|---|---|
| Na₂SO₄ | 0.08500 |
| C₅H₄N₄O₃ | 0.01250 |
| Na₃C₆H₅O₇ × 2H₂O | 0.03600 |
| C₄H₇N₃O | 0.04405 |
| CH₄N₂O | 0.75000 |
| KCl | 0.11540 |
| NaCl | 0.08780 |
| CaCl₂ | 0.00925 |
| NH₄Cl | 0.06330 |
| K₂C₂O₄ × H₂O | 0.00175 |
| MgSO₄ × 7H₂O | 0.05410 |
| NaH₂PO₄ × 2H₂O | 0.14560 |
| Na₂HPO₄ × 2H₂O | 0.04155 |

TABLE 6
Resulting composition of the Martian Medium in terms of metals

| Components | (g/L) |
|---|---|
| Al | 0.000945 |
| Ca | 0.004595 |
| Fe | 0.001305 |
| K | 0.004655 |
| Mg | 0.00079 |
| Mn | 0.000035 |
| Na | 0.0037 |
| P | 0.00009 |
| Si | 0.00368 |
| Ti | 0.000255 |

### 1.3. Growth Experiments for the Identification Optimal Content of MM in the Culture Medium and the Identification of the Effects of Simulated Martian Conditions

Preliminary tests were performed, using growth media consisting of mixtures of MM and Zarrouk's medium (ZM) wherein the volume percentages of MM were equal of 0, 20, 40, 60 and 80% v/v respectively. From these experiments (data not shown) the best growth medium involving the use of MM was identified to be the one containing 40% v/v of MM and 60% v/v of ZM (Mm40). Subsequently, different experiments were performed to assess the effects of operating conditions that simulate the ones of the process to be implemented on Mars. Both the isolated and the synergic effect of all the operating conditions in the process to be realized on Mars were assessed. Table 7 summarizes the experiments performed in this case. These experiments permitted us to identify the feasibility of the invented process. The features common to all these experiments were the following. The batch culture experiments were carried out within transparent vented cap flasks filled to 40 ml. The experiment was set in triplicates with an illumination of 100 μmol m⁻² s⁻¹ on the irradiated surface of the culture flask. The optical density at the beginning of the experiments was about 0.2 at a wavelength of 650 nm.

TABLE 7
Experiments performed to assess the feasibility of the process

| Experiment ID | Medium* | Atmosphere | Gravity |
|---|---|---|---|
| Zm_Air_1 g | Zm | Air | 1 g |
| Mm40_Air_1 g | Zm (60% v) + Mm (40% v) | Air | 1 g |
| Zm_Air_μg | Zm | Air | μg |
| Mm40_Air_μg | Zm (60% v) + Mm (40% v) | Air | μg |
| Zm_CO₂_1 g | Zm | CO₂ | 1 g |
| Mm40_CO₂_1 g | Zm (60% v) + Mm (40% v) | CO₂ | 1 g |
| Zm_CO₂_μg | Zm | CO₂ | μg |
| Mm40_CO₂_μg | Zm (60% v) + Mm (40% v) | CO₂ | μg |

*Zm = Zarriuk's medium, Mm = Martian medium

Cells morphology was examined using magnification of 40 and 100× (Leica DM750) optical microscope interfaced with Leica EC3 digital camera (Leica Microsystems, Wetzlar, Germany), and using the Leica Application Suite (version 3.4.0, Leica Microsystems). All operations were carried out avoiding environmental contamination and under a microbiological safety cabinet. Atmosphere consisted of air and the gravity was equal to 1 g. During experiments, the growth of cyanobacteria was monitored through absorbance microplate reader ELISA (TECAN, Sunrise™, Tecan Trading AG, Switzerland) of the chlorophyll-a optical density (OD) of the culture at 650 nm wavelength. The biomass concentration Cₓ (g L⁻¹) was calculated from OD measurements using the calibration curve Cₓ vs OD, which was obtained by gravimetrically analysis of the biomass concentration of known culture volumes that were previously centrifuged at 4000 rpm for 15 min and dried at 105° C. for 24 h. The pH was measured daily by pH-meter (Basic 20, Crison). The pH was measured daily by pH-meter (XS instrument, Carpi, MO, Italy).

### 1.4. Simulating the Atmosphere in the Martian Dome (Whitley Jar Gassing System)

In order to investigate the possibility to use CO₂ of Mars atmosphere, further experiments were performed growing microalgae in an atmosphere consisting of pure CO₂. To this aim a Don Whitley workstation, capable to provide excellent conditions for the processing, incubation and examination of samples without exposure to atmospheric oxygen, was used. The workstation allows to manipulate samples in a sustainable environment where parameters can be altered to create the required conditions for growing the cultures in presence of ˜100% CO₂ into the jars in just 2 minutes. The obtained A full color touch screen control panel allows the operator to monitor, in real time, that the criteria necessary for the culture's growth has been met. The workstation is connected to both a CO₂ cylinder and a polycarbonate jar. The capacity of the jar is 2.5 L, (Height 24 cm, Diameter 17 cm) being capable to accommodate 8 flasks with a base section of 75 cm². The jar has an in-built fault detector to create an alert if it is leaking.

### 1.5. Simulating Microgravity Conditions

To verify whether microalgae growth and metabolism could be affected by the microgravity conditions achieved in space and Martian conditions further experiments were performed under microgravity conditions at the laboratory of the Departmental of Biomedical Sciences, University of Sassari, Sardinia, Italy. In order to simulate microgravity (μg) a 3D random positioning machine (RPM, Fokker Space, Netherlands) is used. The 3D Random Positioning Machine (RPM) was a micro-weight (‘microgravity’) simulator that is based on the principle of ‘gravity-vector-averaging’, built by Dutch Space (former Fokker Space), Leiden, The Netherlands. The 3D RPM is constructed from two perpendicular frames that rotate independently. The direction of the gravity vector is constantly changed so that the average of the gravity vector simulates a microgravity environment. The 3D RPM provides a simulated microgravity less than 10⁻³ g. The dimensions of the 3D RPM are limited to 1000×800×1000 mm (length×width×height). A mechanical stage can accommodate a maximum of 12 flasks simultaneously, the samples shall be placed not more than 10 cm from the center of rotation, before being placed in the 3D RPM.

### 1.6. Simulating the Simultaneous Effect of all the Operating Conditions of the Process to be Implemented on Mars

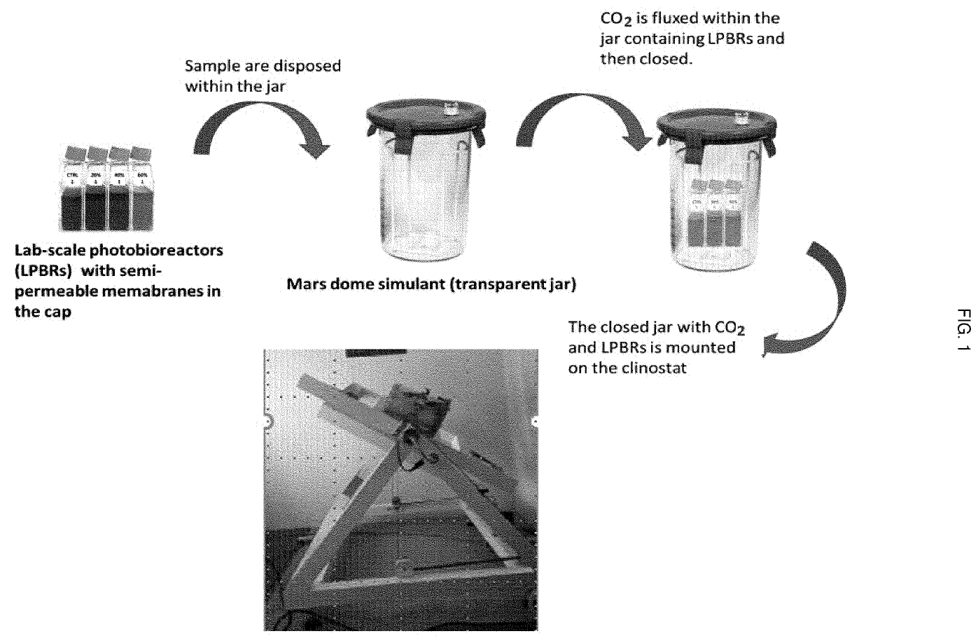

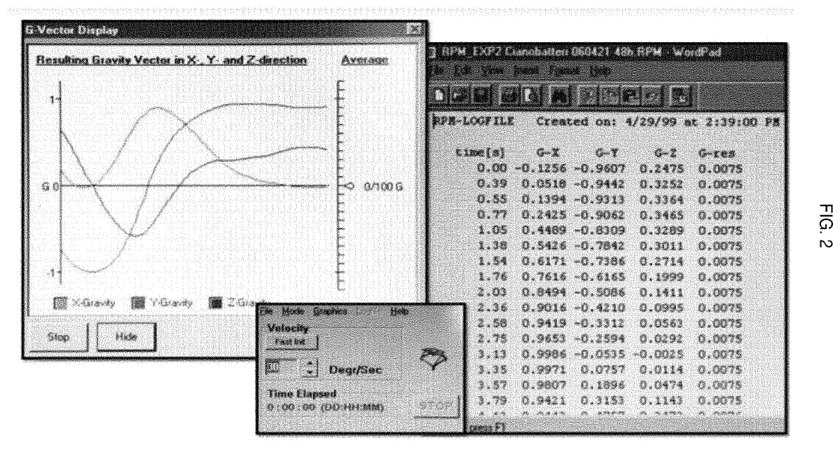

To simulate the effect of all the operating conditions of the invented process on Mars, the following procedure was adopted. The flasks were carefully filled (approximately 80 ml) with medium a containing 40% v/v of MM (Mm40) without air bubbles to avoid shearing of the fluid. Subsequently, the flasks were fixed inside the jar, filled with CO₂, that in turn was mounted on the 3D RPM. The latter one was operated for at least 23 days in a dedicated room at 25° C. The same cultures were grown in parallel at 1 g, comprised the control cultures and placed in the static bar to undergo the same vibration of the sample in μg conditions. The 3D RPM is connected to a computer and through a specific software the mode and speed of rotation were selected. Random Walk mode with a 60 degree/sec (rpm) was chosen. The cultures were enlightened with a white illumination of 150 μmol m⁻² s⁻¹ for 12 hours and CO₂ has been administered to the microalgae while the light hours. A simplified scheme of the experiment's steps is shown in FIG. 1 while FIG. 2 shows a graph of the vectorial components of the acceleration achieved by the RPM and a table where the vectorial sum (G-res) of such components (whose values are always close to zero) is reported.

### 2. RESULTS OF EXPERIMENTS

### 2.1. Isolated Effect of the Use of Martian Medium

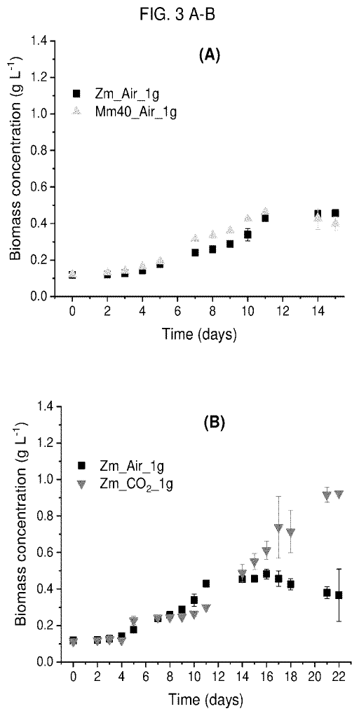

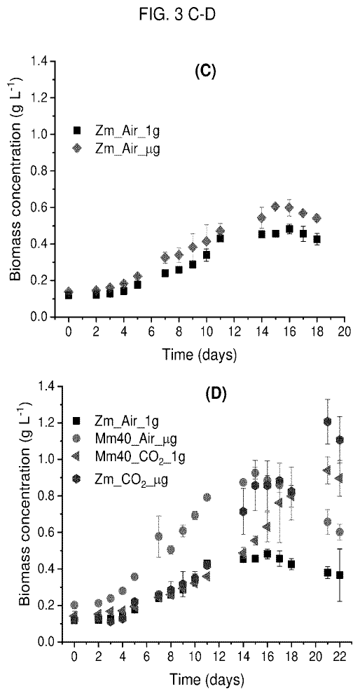

This experiment was aimed to verify whether the replacement of a volume of Zarrouk's medium with the same volume of Martian medium could affect the growth of Spirulina. To this aim preliminary tests were performed, using growth media consisting of mixtures wherein the volume percentages of Mm were equal of 0, 20, 40, 60, 80% v/v and 100% respectively. These experiments were performed using atmospheric air and earth gravity. From these experiments (data not shown) the best growth medium involving the use of Mm was identified to be the one containing 40% v/v of Mm and 60% v/v of Zm (Mm40). Higher percentages resulted in a reduction of the growth rate of the culture. In FIG. 3A the comparison between the biomass concentration evolution obtained when using only Zm and Mm40 is shown. No relevant effect resulted from the re-placement of 40% v/v of Zm with a corresponding volume of Mm up to 14 days when both the cultures achieved a concentration close to 0.45 g L⁻¹. Rather a slight improvement of the growth could be observed up to 13 days of cultivation. After the 15th day of cultivation both cultures start to decrease (data not shown) probably due to carbon starvation or the inhibition determined by the high pH achieved by the systems (close to 11). So, in order to be productive when operated under batch mode, these cultures should be stopped after 15 days of cultivation.

### 2.2. Isolated Effect of the Use of Simulated Martian Atmosphere

The goal of this experiment was to isolate the effect of using an atmosphere that simulates the one that will be realized in the domes hosting the process of the present invention on Mars. According to the process of the invention, the latter one will be obtained from the Mars atmosphere and will consist of almost pure CO₂ pressurized at a pressure equal to at least 0.8 bar. For this reason, these experiments were performed by inserting the laboratory scale photobioreactors (flasks) containing the microalgal cultures within a jar wherein pure CO₂ was then fluxed until its partial pressure within the jar was equal to 1 bar. The obtained results are shown in FIG. 3B. In this case the medium was always Zarrouk's medium. No relevant effect resulted from the re-placement of air with CO₂ simulating Martian atmosphere in the Martian dome simulant could be observed up to the 14^{th} day of cultivation. However, from that moment on, the culture using the simulated Martian atmosphere, i.e. CO₂, kept on growing while the biomass concentration of the culture grown in air started to decrease. This demonstrate that from the 14^{th} day, carbon represent the main limiting factor for Spirulina growth. Therefore, the use of an atmosphere simulating the one foreseen in the proposed process is not only feasible but even advantageous with a consequent reduction of the payload needed to bring CO₂ cylinders on Mars.

### 2.3. Isolated Effect of Simulated Microgravity

This experiment was aimed to evaluate how simulated microgravity could affect the growth of Spirulina Platensis. Hence, it was performed by mounting the flasks containing culture of Spirulina in Zarrouk's medium on the Random Positioning Machine and monitoring growth periodically. The corresponding results are shown in FIG. 3C. A slight improvement of the growth rate was observed when cultivating the sample under microgravity conditions. This might be due to a reduced effect of settling and aggregation determined by the gravity. The latter phenomena might in fact hinder the diffusion of nutrients to the algae. Ultimately, although the gravity on Mars is slightly higher than the one adopted in this experiment, the latter demonstrates that reduced gravity conditions taking place on Mars not only don't affect the growth of Spirulina but can even improve slightly its growth.

### 2.4. Synergic Effects of the Combination of Two Out of Three of the Operating Conditions of the Process of the Invention to be Implemented on Mars

The goal of these experiments was to explore the effect of the simultaneous application of two out of three operating conditions simulating the ones taking place in the process to be realized on Mars, for example Martian atmosphere plus microgravity, Martian medium plus microgravity or Martian medium plus Mars atmosphere, etc. FIG. 3D shows that all the possible combinations of operating conditions resulted in synergic effect providing a better growth of microalgae when compared to the base case experiment (Zm_air_1 g). In particular, up to the 15th day of growth the culture using Martian medium under microgravity (Mm40_Air_μg) was the better one but form the 16th day on it started to decrease probably due to the lack of carbon. On the contrary the two cultures using CO₂, i.e. the simulated Martian atmosphere used in the process, kept on growing for all the experiment duration thus demonstrating the capability of this strain to benefit of the high carbon concentration in the liquid. The best performance was then obtained when CO₂ and microgravity was used (Zm_CO₂_μg). In fact, in the latter experiment a biomass concentration of about 1.2 g L⁻¹ was achieved after 22 days of cultivation.

### 2.5. Simulating all the Operating Conditions Taking Place on Mars According to the Process of the Invention

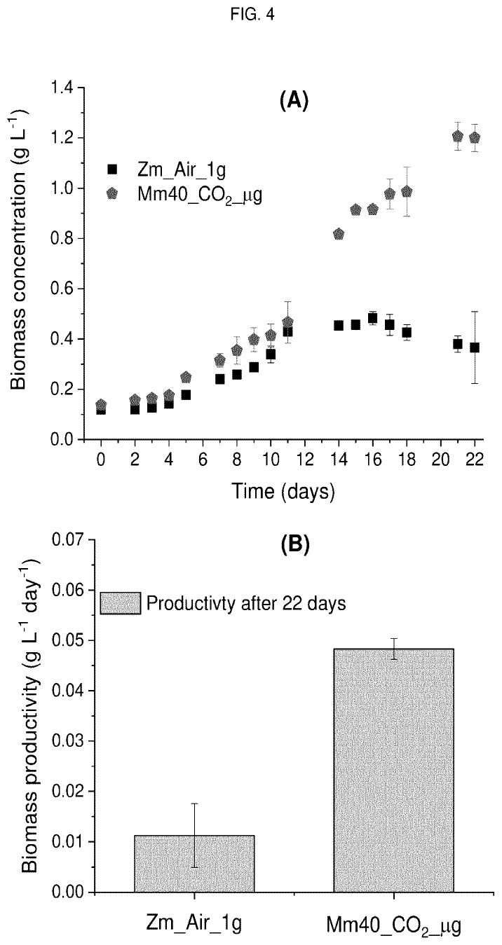

In this experiment all the operating conditions of the invented process taking place on Mars, i.e. microgravity, CO₂ atmosphere and Mm40, are simultaneously applied to verify its feasibility. The obtained results are shown in FIG. 4A.

It can be observed that the simultaneous use of all the operating conditions of the process not only did not affect the cultivation of microalgae but also resulted in a relevant improvement of their growth showing a synergic effect. This is probably due to the fact that when the biomass concentration becomes high (at the end of the experiment) the culture requires more CO₂ to perform photosynthesis while microgravity conditions avoid aggregation and settling. Moreover, the strain Spirulina well tolerate high CO₂ concentrations because its photosynthesis is capable to significantly increase culture pH and counteract the acidification effects of CO₂ which potentially inhibit growth. While the reasons underlying such an improvement should be better investigated in further studies the experimental evidence is univocal and further confirmed by the comparison of the productivities achieved by the two curves after 22 days (cf. FIG. 4B).

Ultimately, based on these results, it can be reasonable stated that, although the gravity on Mars is slightly higher than the one adopted in this experiment, the process of the invention is advantageously feasible allowing the production of food and oxygen on Mars. The possibility to use in-situ available resource such as regolith and atmosphere would lead also to a relevant reduction of the payload of the kit transferred from Earth to Mars to implement the process in-situ.

### 2.6. Comparison the Performances of the Process of the Present Invention with Respect to the One Described in WO2013014606

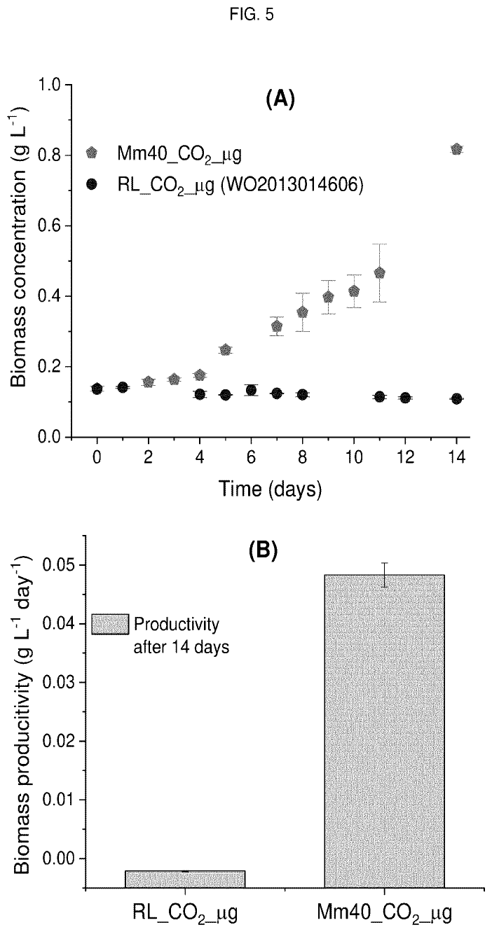

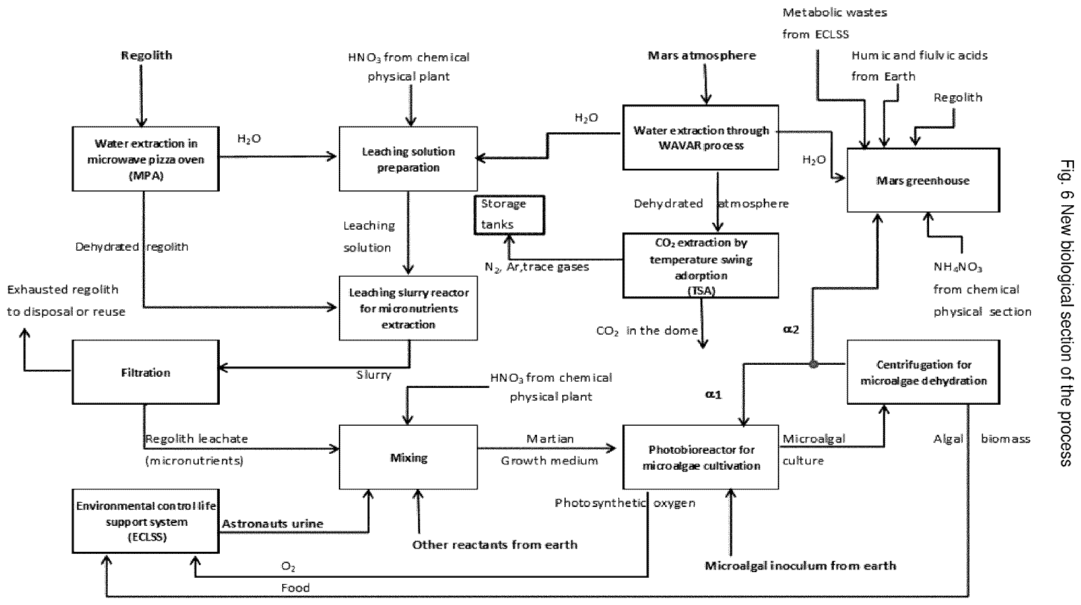

In order to assess the improvement of the process of the present invention with respect to the state-of-the-art related to microalgae cultivation in extra-terrestrial contexts by exploiting the bio-ISRU paradigm, a further experiment was carried out under the operating conditions described in WO2013014606. In the latter one, growth medium for microalgae consisted of regolith leachate enriched with HNO₃ (RL) so to provide the nitrates needed by microalgae to grow. So, in this experiment the growth medium was obtained by adding to the leachate HNO₃ so to obtain a final nitrate concentration equal to the one of the Z-medium. For the rest, the operating conditions were kept equal to the ones of the present invention, i.e. atmosphere consisting of pure CO₂ and microgravity. The comparison between the results obtained with the configuration of the process of the present invention (Mm40_CO₂_μg) and the one of WO2013014606, called RL_CO₂_μg, is shown in FIG. 5A in terms of biomass concentration evolution with time and in FIG. 5B in terms of biomass productivities achieved after 13 days of cultivation. It can be seen that, when using the operating conditions reported in WO2013014606 (experiment RL_CO₂_μg) no biomass concentration increase could be detected up to the 14th day of cultivation. On the contrary, a slight decrease could be observed. Accordingly, while the algae were capable to survive under the conditions reported in WO2013014606 (because the decrease wasn't relevant) they weren't capable to grow and replicate significantly. Therefore, the corresponding productivity after 14 days was slightly negative but close to zero (cf. FIG. 6B). For this reason, this experiment was stopped after 14 days. Therefore, the comparison with the results obtained with the invented process (Mm40_CO₂_μg) are reported up to 14 days. As it can be seen the biomass productivity is very much higher when using the operating conditions of the process of the present invention. This evidence clearly demonstrates the relevant improvement provided by the present invention with respect to the state of the art.

1. An apparatus for simulating on Earth the growth of a cell line under extra-terrestrial conditions, said apparatus comprising:
a insulating jar mounted on a 3D clinostat or random positioning machine (RPM), said jar capable of containing at least one laboratory scale bioreactor (LSB) which contains the cell line to be cultivated together with a culture medium which is as close as possible to the optimal one and simulating when possible In Situ Resource Utilization (ISRU), said jar provided with a manometer capable to measure the pressure inside the jar, a gas inlet and a gas outlet; and
a cylinder for storing a gas simulating an extra-terrestrial atmosphere, the cylinder having an outlet fluidly connectable with the inlet of the jar.

2. The apparatus according to claim 1 wherein the 3D clinostat or RPM is programmed to bestow the jar a motion characterized by a resulting acceleration vector, whose module has an average value over time that is close to zero or as close as possible to the gravity conditions of the extra-terrestrial location which is object of the simulation.

3. The apparatus according to claim 1, wherein the jar is transparent and the LSB is a transparent Laboratory Scale Photobioreactor (LSP).

4. The apparatus according to claim 3 wherein the at least one LPS contains at least one algal strain selected from the group consisting of:
Gloeocapsa strain OU_20, Leptolyngbya strain OU_13, Phormidium strain OU_10, Chroococcidiopsis 029; Arthrospira platensis; Synechococcus elongatus; Anabaena cilindrica; Chlorella vulgaris; Nannochloris Eucaryotum and genetically engineered strains thereof.

5. The apparatus according to claim 1, wherein the at least one LSB contains a cell line selected from in the group consisting of Chlorella sorokiniana, Chlorella zofigensis, Coccomyxa sp., Synechococcus sp., Pseudochloris wilhelmii, Chlorella protothecoides, Euglena gracilis, Chlamydomonas reinhardtii, Isochrysis galbana, Neochloris oleoabundans, Scenedesmus obliquus, Dunaliella salina, Nannochloropsis oculate, Chlorella pyrenoidosa, Botryococcus braunii, Phaeodactylum tricornutum, Tetraselmis sp., Thalassiosira pseudonana, Haematococcus pluvialis, Nannochloropsis oceanica, Spirulina maxima, Pavlova salina, Porphyridium marinum, Tetraselmis inconspicua, Cyanophora paradoxa, Thalassiosira rotula, Amphora sp., Odontella aurita, Attheya sp., Chromulina ochromonoides, Diacronema vlkianum, Chaetoceros sp., Navicula pelliculosa, Odontella mobiliensis, and Porosira pseudodenticulata; or from in the group consisting of H1, H9, Embryonic stem cells, Human; HEK-293, Embryonic kidney transformed with adenovirus, Human; HeLa, Epithelial cell, Human; HL 60, Human, promyelocytic leukemia cells, Human; MCF-7 Breast cancer, Human; A549, Lung cancer, Human; A1 to A5-E, Amnion, Human; ND-E, Esophagus, Human; CHO, Ovary, Chinese hamster; 3T3, Fibroblast, Mouse; BHK21, Fibroblast, Syrian hamster; MDCK, Epithelial cell, Dog; E14.1, Embryonic stem cells (mouse), Mouse; COS, Kidney, Monkey; DT40, Lymphoma cell, Chick; S2, Macrophage-like cells, Drosophila; GH3, Pituitary tumor, Rat; L6, Myoblast, Rat; Sf9 and Sf21, Ovaries, Fall Army worm; (Spodoptera frugiperda) ZF4 and AB9 cells, Embryonic fibroblast cells, Zebrafish; 1184, skin fibroblast, Human; E6.1 clones, Jurkat cell, Human; THP1 cells, Human; SH-SY5Y, Neuroblastoma cells, Human; iPSCs, stem cells, Human; Erythrocyte cell culture, Human; C20A4, Chondrocyte cell, Human; 1301, T-cell leukemia, Human; 1306, 161BR, skin fibroblast, Human; F-36P myelodysplastic syndrome, leukemia, Human; H9, T-cell, Human; HeLa, Epithelial cell, Human; E6.1 clones, Jurkat cell, Human; SH-SY5Y, Neuroblastoma cells, Human; iPSCs, stem cells, Human; 1184, skin fibroblast, Human; hMSCs, mesenchymal stem cells, Human; mBMSC, Bone Marrow derived mesenchymal stem cell, Rat; ADSCs, Adipose-derived stem cell, Human; mESCs, Embryonic stem cells, Mouse; MG-63, Osteosarcoma cell lines, Human; HUVEC, and Human umbilical vein endothelial cells, Human.

6. A method of simulating on Earth the cellular growth under extra-terrestrial conditions on a pre-determined extra-terrestrial location, said method comprising using the simulation apparatus according to claim 1.

7. The method according to claim 6 comprising:
preparing a culture broth culture broth which is as close as possible to the optimal medium for the cell line to be grown, and when possible by simulating extra-terrestrial ISRU;
loading the LSP of the apparatus of the invention with the culture broth and successively with the inoculum of a microalgae or cyanobacteria strain to be cultivated;
arranging the LSP within the jar;
mounting the jar on the clinostat or RPM of the apparatus of the invention;
connecting the outlet of the cylinder of the apparatus of the invention with the gas inlet of the jar;
insufflating the extra-terrestrial atmosphere simulant into the jar with the gas outlet of the jar open for a time sufficient to wash the jar inner atmosphere;
closing the jar outlet while keeping insufflating the extra-terrestrial atmosphere simulant until an internal pressure of at least 0.8 bar is achieved within the jar; and
switching on the clinostat or RPM to simulate microgravity.

8. The method according to claim 6, for the cultivation of an edible microorganism further comprising preparing a culture medium by mixing a liquid regolith leachate obtained by leaching with acidified water an extra-terrestrial regolith simulant, with a diluted astronaut's urine simulant and micronutrients, wherein the micronutrients are those unavailable by ISRU on the extra-terrestrial location and are known to be essential for the growth of the strain to be cultivated.

9. The method according to claim 8 comprising the following steps:
a) preparing an extraterrestrial regolith simulant;
b) contacting the extraterrestrial regolith simulant with a leaching solution to obtain a regolith slurry; the leaching solution being water acidified with HNO₃;
c) filtrating the regolith slurry to obtain a solid exhaust-regolith and a liquid regolith leachate.
d) preparing an astronaut's urine simulant;
e) diluting the urine simulant with water in order to simulate the dilution determined by flushing-water in most ECLSS. This way a ECLSS wastewater simulant is obtained;
e′) eventually further diluting the wastewater simulant if its salinity is too high to be compatible with microalgae growth;
f) preparing a culture medium with the optimal micronutrients for the growth of the strain to be cultivated;
g) mixing the ECLSS wastewater simulant with the regolith leachate and the culture medium to obtain a culture broth;
h) loading the LSP of the apparatus of the invention with the culture broth and successively with the inoculum of a microalgae or cyanobacteria strain to be cultivated;
i) arranging the LSP within the jar of the apparatus which simulates an extra-terrestrial dome;
j) mounting the jar on the clinostat or RPM of the apparatus of the invention;
k) connecting the outlet of the cylinder of the apparatus of the invention with the gas inlet of the jar;
l) insufflating the extra-terrestrial atmosphere simulant into the jar with the gas outlet of the jar open for a time sufficient to wash the jar inner atmosphere;
m) closing the jar outlet while keeping insufflating the extra-terrestrial atmosphere simulant until an internal pressure of at least 0.8 bar is achieved within the jar; and
n) switching on the clinostat or RPM to simulate microgravity and simultaneously irradiating the dome simulant with natural or artificial light to promote photosynthesis.

10. A bio-ISRU process for producing photosynthetic edible biomass and oxygen for sustaining long-term manned extra-terrestrial missions;
said process comprising:
preparing an extra-terrestrial growth medium by mixing a regolith leachate with diluted astronaut urines coming from a ECLSS, and other micronutrients unavailable in situ brought from Earth which are essential for the growth of the edible biomass; and
loading a photobioreactor with the extra-terrestrial growth medium and with an inoculum of the edible biomass brought from earth.

11. The process according to claim 10 comprising the following steps:
a′. assembling on the extra-terrestrial soil at least one geodesic dome and place at least one photobioreactor within the dome;
b′. assembling a physico-chemical section comprising photovoltaic panels, at least a WAVAR unit, at least a TSA unit and at least a MPO unit for extracting from extra-terrestrial soil and atmosphere water, dehydrated and pressurized CO₂, N₂ and Ar and producing by NH₃, O₂, H₂, HNO₃, NH₄NO₃;
c′. blowing heated, pressurized and dehydrated CO₂ produced in the step (b′) within said dome until a pressure of at least 0.8 bar and temperature of at least 10° C., preferably between 1° and 15° C., is reached within the dome;
d′. preparing a leaching solution by mixing water and HNO₃ produced in the physico-chemical section;
e′. leaching the dehydrated regolith from the physico-chemical section with the leaching solution, preferably with a solid/liquid weight ratio of 1:5 for at least one Martian day (sol);
f. filtering the regolith slurry to obtain a regolith leachate and leached regolith;
g′. preparing an extra-terrestrial growth medium by mixing a regolith leachate with diluted astronaut urines coming from a at least one ECLSS section, HNO₃ produced in the physico-chemical section and other micronutrients unavailable in situ brought from Earth which are essential for the growth of the edible biomass;
h′. preparing an inoculum of the edible microalgae or cyanobacteria brought from Earth;
i′. feeding the photobioreactor with the extra-terrestrial growth medium and successively with the inoculum to obtain a biological slurry;
j′. exposing the biological slurry to the CO₂ within the dome and to a light source which is capable of promoting photosynthesis, thus leading to the formation of new biomass algal and photosynthetic oxygen;
k′. separating the algal biomass from the spent “culture broth” by centrifugation and extracting photosynthetic oxygen by degassing;
l′. sending the oxygen to the ECLSS section and further dehydrating the algal biomass in order to use it as food or dietary supplement along with the food produced in the ECLSS section;
m′. splitting the spent “culture broth” into two streams named α₁ and α₂;
n′. recirculating the stream of the spent culture broth α₁ into the at least one photobioreactor; and
o′. optionally conveying the stream α₂, together with the ammonium nitrate (NH₄NO₃) produced in the physico-chemical section, together with fresh regolith, together with suitable amounts of humic and fulvic acids brought from Earth, together with human metabolic wastes from ECLSS, into the domes where vegetables are grown.

12. A food for astronauts comprising the edible biomass obtained by the process according to claim 10.

13. A kit of material specifically adapted for implementing the process according to claim 10 during a long-term manned space mission; said kit of material comprising:
a system for conveying diluted astronaut urines coming from a ECLSS to a container for preparing the extra-terrestrial growth medium; and
the micronutrients which are essential for the growth of the edible biomass and are unavailable in the extra-terrestrial location.

14. The kit according to claim 13 further comprising:
at least one geodesic dome for housing the different plant units used in the physico-chemical group of the procedure above;
at least one photovoltaic system for producing the energy needed for heating the inner atmosphere of the at least one dome, as well as the energy needed for powering plant units operation;
at least one WAVAR unit based on the use of zeolites through which adsorption processes, followed by desorption through microwaves heating, are performed for the extraction of water from Martian atmosphere;
at least one TSA unit consisting of at least one adsorbent bed of zeolite and at least one radiator that ensure the heat exchange with Martian environment and the implementation of adsorption-desorption cycles at variable temperature that, in turn, allow the separation of CO₂ from other gases constituting the Martian atmosphere (mainly N₂ and Ar) as well as its pressurization. Pressurized pure CO₂ produced by TSA units can be the blown within the at least one dome until a suitable pressure is achieved in the inner of the dome;
at least one excavator and at least one conveyor belt for excavating and conveying the Martian regolith to the following treatment units;
at least one MPO unit, including at least one magnetron, for the extraction of adsorbed and hydration water from the Martian regolith by microwave heating;
at least one unit for mixing the water extracted from the regolith with suitable amounts of nitric acid produced in the physico-chemical section;
at least one leaching reactor operating in continuous mode, for leaching regolith through a mixture of water and nitric acid;
at least one unit consisting of a “filter plates” for the solid/liquid separation of the slurry stream outgoing from the leaching reactor. Said unit produces a liquid stream named “regolith leachate” and a solid stream of “leached regolith”;
at least one unit for mixing the “regolith leachate” with the urine diluted with flushing water produced by astronauts in the ECLSS to obtain the so called “culture broth”;
at least a tank for storing the gas, consisting mainly of N₂ e Ar, which has been produced in the TSA unit as a result of CO₂ separation;
at least one algal strain selected in the group consisting of: Gloeocapsa strain OU_20, Leptolyngbya strain OU_13, Phormidium strain OU_10, Chroococcidiopsis 029; Arthrospira platensis; Synechococcus elongatus; Anabaena cilindrica; Chlorella vulgaris; Nannochloris Eucaryotum or genetically engineered strains thereof;
at least one unit for preparing the inoculus of algal strains;
at least a photobioreactor for producing algal biomass;
nutrients from Earth, said nutrients those unavailable by IRSU but essential for the growth of the at least one algal strain and;
at least one unit for the separation of algal biomass and of oxygen produced in the photobioreactor from the spent “culture broth”;
at least one unit for dehydrating algal biomass; and
optionally at least one geodesic dome to be used as a greenhouse for growing edible plants.
